(19) United States
(12) Patent Application Publication
(10) Pub. No.: US 2004/0175351 A1
(43) Pub. Date: Sep. 9, 2004

(54) Compositions and methods for reducing or controlling oxidants and aging

(76) Inventors: Kay Liu, Monrovia, CA (US); Craig Miller, Diamond Bar, CA (US)

Correspondence Address: Craig Miller, 23719 Jayhawker, Diamond Bar, CA, 91765, US

(21) Appl. No.: 10/377,551

(22) Filed: Mar. 3, 2003

(51) Int. Cl. A61K 35/78

(52) U.S. Cl. 424/74; 424/729

(57) ABSTRACT

The present invention provides the protection of the human skin or other biological membrane material. In the method of using a chemical system, oxidant and free radical particles are naturalized thus protecting the skin from damage.

### FIELD OF THE INVENTION

The present invention relates to the protection of the human skin or other biological membrane material. In the method a chemical system is delivered to the layers of the skin by use of a chemical system. Upon delivery, the chemical system works to deoxidize the dermal layers of human skin.

### BACKGROUND OF THE INVENTION

The benefits of antioxidants are well known in the art of practicing medicine. In general, during respiration, on a cellular level, some oxygen molecules are converted into what are called ‘free radicals’, i.e. superoxides, hydrogen peroxide, and single oxygen atoms. These ‘free radicals’ are unstable molecules and will react with other compounds, which results in the stabilization of the free radicals. Unfortunately, this stabilizing process can be damaging to normal, healthy tissues and/or can cause changes to cellular DNA resulting in mutations, which can cause cancer.

Antioxidants are molecules that interact with the free radicals thereby neutralizing them, which results in protecting normal tissue and DNA from potential damage. Once the antioxidants have neutralized the free radicals, they are either recycled or remain in a stable, altered state. Normal cellular respiration is not the only source of free radicals. Our environment exposes the body to a number of additional free radical sources, i.e. ionizing radiation (industry, the sun, cosmic rays), ozone and nitrous oxide primarily from auto exhaust emissions, heavy metals (lead, mercury, and cadmium), cigarette smoke both primary and secondary, and alcohol. When these free radicals enter the body, they can react with healthy tissues the same as the free radicals that are the result of cellular respiration, again with the potential to cause damage. It is believed that free radicals play a role in many different disease processes by causing oxidative stress.

It is known in the art that Tea's are an excellent antioxidant because tea is apolyphenols, which belongs to the class of compounds known as flavonoids. Flavonoids, are comprised of a number of different compounds such as catechins, flavanones, flavonols, isoflavones, chlorogenic acid, coumarylquinic acid, and one that is unique to tea, theogallin (3-galloylquinic acid). Flavonoids are substances of plant origin that have flavone, a plant pigment, as a base in various combinations and with varying beneficial properties. Other flavonoids include quercetin, quercitrin, rutin, and hesperidin. In oxidized tea, black and oolong, there is less of the catechins and there are other compounds, formed during oxidation, that negatively react to catechins such as theaflavins, thearubigins, bisfiavanols, and epitheaflavic acids. There have been many attempts to use white tea which is non-oxidized tea thus retaining more catechins without adding other negative compounds.

### SUMMARY OF THE INVENTION

The present invention provides a chemical system for preventing the oxidization and free radicals from damaging the skin.

The primary parts of the chemical system include white tea extract and vitamin C. White Tea is a green tea leaf that has not matured. Like other tea, white tea is an unfermented and non-oxidized tea. White tealeaves are quickly and completely dried after harvest to prevent fermentation and oxidation and permit a purer, more natural state. This process is known in the art to allow a greater concentration of polyphenols. Polyphenols in non-oxidized tea are catechins. The four main catechins are epicatechin (EC), epicatechin gallate (ECG), epigallocatechin (EGC), and epigallocatechin-3-gallate (EGCG). Of all of these compounds, EGCG comprises about 60% and it also has been shown to be the main physiologically active polyphenol in non-oxidized tea.

The liquid form of Vitamin C, L-ascorbic acid (L-3-Ketothreohexuronic acid lactone) is also a good antioxidant. Vitamin C can be broken down particles sizes between 5 μm to 200 μm, which helps ease and penetrate the pores of the epidermis layer of the skin and allows vitamins and minerals to pass to a deeper layer of the skin, the dermis layer. It is at the dermis layer where oxidized particles and free radicals can be stored and cause the greatest damage to the dermis layer of the skin.

### BRIEF DESCRIPTION OF THE DRAWINGS

FIG. 1 illustrates the layers of the skin

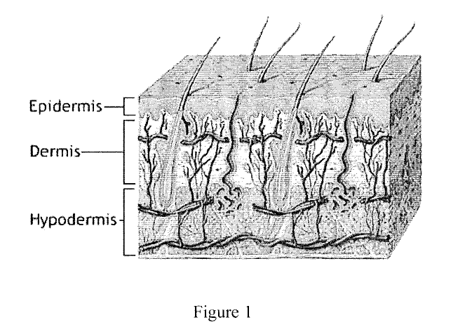

### DETAILED DESCRIPTION OF PREFERRED EMBODIMENTS

The preferred embodiment of this chemical system, which includes vitamin C and white tea, is practiced when the chemical system is applied to the epidermis layers of the skin. Upon application of the chemical system the vitamin C helps ease the pores of the epidermis layer of the skin, which allows vitamins and minerals with very small particle sizes to pass to a deeper layer of the skin, the dermis, layer. It is at the dermis layer where oxidized particles and free radicals can be stored and cause the greatest damage.

Once the vitamin C and catechins have reached the dermis layer of the skin, the catechins interact with the free radicals thereby neutralizing them, which results in protecting normal tissue and DNA from damage.

What is claimed:

1. A revitalizing human skin system comprising:
a white tea that is formulated from green tea leaves; and
a liquid form of vitamin C, which is, composed of particles sizes between 5 μm to 200 μm.

2. The revitalizing human skin system of claim 1, wherein the white tea is a green tea leaf that has not matured.

3. The revitalizing human skin system of claim 1, wherein the liquid form of vitamin C is L-ascorbic acid (L-3-Ketothreohexuronic acid lactone).

4. A method of revitalizing human skin comprising the steps of
applying a liquid form of vitamin C and the white tea extract to the skin.

5. A method of revitalizing human skin of claim 4 wherein the liquid forms of vitamin C is L-ascorbic acid (L-3-Ketothreohexuronic acid lactone).
